(19) United States
(12) Patent Application Publication
(10) Pub. No.: US 2023/0201058 A1
(43) Pub. Date: Jun. 29, 2023

(54) PROTECTIVE BARRIER FOR USE IN PHYSICAL EXAMINATIONS, DIAGNOSTICS AND TREATMENTS AND RECREATION

(71) Applicant: Debora Ruth VILCOVSKY SOKOLOW, Tel Aviv (IL)

(72) Inventor: Elias VILCOVSKY, Tel-Aviv (IL)

(73) Assignee: Debora Ruth VILCOVSKY SOKOLOWER, Tel Aviv (IL)

(21) Appl. No.: 18/008,214

(22) PCT Filed: Jun. 3, 2021
(86) PCT No.: PCT/IL2021/050669

§ 371 Date: Dec. 5, 2022

(30) Foreign Application Priority Data
Jun. 4, 2020 (IL) 275142

(51) Int. Cl. A61G 10/00; A61B 42/00

(52) U.S. Cl. CPC A61G 10/005; A61B 42/00

(57) ABSTRACT

A barrier for use in one or more of physical examinations, diagnostics, treatments and recreation includes one or more panels. At least one of the panels includes a window through which at least one of a physical examinations, diagnostic procedure and treatment may be performed. The barrier includes at least one structural feature that is configured to reversibly collapse the barrier into a compact configuration while not in use or for transporting the barrier.

### RELATED APPLICATION/S

This application claims the benefit of priority of Israel Patent Application No. 275142 filed 4 Jun. 2020, the contents of which are incorporated herein by reference in their entirety.

### FIELD AND BACKGROUND OF THE INVENTION

The present invention, in some embodiments thereof, relates to a barrier for use in physical examinations, diagnostics and treatments and more particularly, but not exclusively, to a barrier that protects caregivers from contracting COVID-19 virus during physical examinations, diagnostics and treatments.

While examining a patient suspected of having a contagious infectious disease, there is a concern that the caregiver will be infected. This concern has grown in the wake of the COVID-19 pandemic. Data from published epidemiology and virologic studies provide evidence that COVID-19 is primarily transmitted from symptomatic people to others who are in close contact through respiratory droplets, by direct contact with infected persons, or by contact with contaminated objects and surfaces.

During a pandemic, the number of patients seeking medical care can vary greatly and quickly. Some of the patients may potentially be infected. Furthermore, some of the medical equipment may be infected. Medical facilities must adapt quickly so that the caregivers, e.g. doctors may continue to provide care while being protected against contracting the virus. During a pandemic, medical staff and the general public may be faced with a lack of available protective gear and disinfectants.

For pandemics especially those that spread by close contact, direct contact and/or indirect contact, e.g., by respiratory droplets, adaption of the medical facility, becomes challenging. Often medical staff is required to wear protective garments to avoid being infected. The importance of properly wearing and removing the protective garments has been discussed in length.

### SUMMARY OF THE INVENTION

According to an aspect of some example embodiments there is provided a collapsible barrier, e.g. wall or screen that includes one or more sleeves through which a caregiver can perform a physical examination, diagnostics and/or physical treatment. The physical examination, diagnostics and/or treatment may be performed by hand and/or with the aid of a device, e.g. stethoscope, ultrasound transducer, dental equipment, autoscope, X-ray machine, other medical instrument, tool, imaging device or a swab. Optionally, the collapsible barrier includes one or more sleeves through which a caregiver can perform a blood test, e.g. extract blood from a patient. The caregiver can be medical personnel or any person that provides treatment including for example cosmetic treatments or massages. Optionally, the collapsible barrier includes one or more sleeves through which a caregiver can provide cosmetic treatment and/or hair treatment, e.g. a haircut.

According to some example embodiments, the barrier is configured to be easily erected when needed and easily collapsed and stored while not in use. Optionally, the barrier has a compact configuration in its collapsed state. Optionally, a dedicated cover is configured to cover the barrier while stored in its collapsed state. Optionally, the barrier is configured to be lightweight so that it can be easily transported to a desired location as needed. Optionally, the barrier includes wheels, or other mechanism for sliding the barrier and may not be required to be lightweight.

According to some example embodiments, the barrier is formed from a single panel that is collapsible, e.g. foldable or rollable. In some example embodiments, the barrier is configured to be modular. Optionally, modular sections that can be selectively combined to construct a barrier in a desired configuration. Optionally, the barrier includes one or more openings and/or windows through which an examination, diagnostic procedure and/or a treatment may be performed. In some example embodiments, one or more windows are fitted with a sleeve through which caregiver can insert a hand or a device for examining and/or treating a without exposing the caregiver or care receiver to respiratory droplets or potential infection by direct and indirect contact. The sleeve may be formed from flexible material. Optionally, the sleeve is formed from elastic material. Optionally, a disposable glove can be worn on the elastic sleeve. In some example embodiments, one or more windows are fitted with a sleeve through which care receiver, e.g. a patient can extend a hand, arm, leg or other body part to receive an examination, diagnostics and/or treatment. Optionally, a same sleeve may be used by the caregiver to extend a hand or a device and may also be used by the care receiver to extend a hand or other body part.

Optionally, the sleeve is a glove or a glove including an arm. Optionally the sleeve is adapted to insert a stethoscope. Optionally, the sleeve has an open end through which an instrument or device may be inserted. Optionally, the sleeve has an opening configured to expose a portion of a care receiver's body for example taking a blood sample, providing blood transfusion and/or sampling discharge from a specific area, e.g. a wound.

According to some example embodiments, the barrier can have a number of windows in different locations and having different sizes and shapes. Optionally, the barrier may include a collapsible shelf, hooks or other structures to facilitate use by the caregiver. Optionally, one or more windows may be configured for installing a microphone, an air purifier, ventilation and light fixture.

In some example embodiments, the barrier is configured to be versatile and adaptable to different needs. Different needs may include for example different levels of protection, different types of examinations and/or treatments as well as different physical setups. In some example embodiments, the sleeves in one or more of the windows may be replaceable and optionally disposable. The barrier may be suitable for medical examinations, diagnostics and treatments as well as recreational treatments, e.g. massage and cosmetic treatments including manicures, pedicures, and hair salon care.

In some example embodiments, the barrier may enable the caregiver to perform examination and/or treatment without wearing protective clothing and a mask. Often, protective clothing and masks are disposable. Reducing the need for using such disposable items is beneficial to the environment and may also reduce medical cost Furthermore, the need to carefully wear and strip off the protective garment after use is both time consuming and cumbersome for the caregiver. In addition, the caregiver may be able to operate in a more comfortable manner while not wearing additional items for protection.

According to an aspect of some example embodiments, there is provided a barrier for use in one or more of physical examinations, diagnostics, treatments and recreation, the barrier comprising one or more panels, wherein at least one of the panels includes a window through which at least one of a physical examinations, diagnostic procedure and treatment may be performed, wherein the barrier includes at least one structural feature that is configured to reversibly collapse the barrier into a compact configuration while not in use or for transporting the barrier.

Optionally, the at least one structural feature is a fold line in the panel and wherein the panel of the one or more panels is configured to be folded along fold lines in the panel.

Optionally, the at least one structural feature is a hinge connecting a pair of panels of the one or more panels and wherein the barrier is configured to fold along the hinge connection.

Optionally, the at least one structural feature is a flexible strap connecting a pair of panels of the one or more panels and wherein the barrier is configured to fold along the flexible strap.

Optionally, the at least one structural feature is a locking mechanism and wherein the one or more panels are modular elements are configured lock onto one another with a removable locking connection.

Optionally, the barrier includes a seal configured to seal a gap in a connection between the one or more panels.

Optionally, the barrier includes a handle fixedly attached to at least one of the one or more panels based on which the barrier may be manually handled.

Optionally, the barrier includes at least one of a hook and a shelf fixedly attached to at least one of the one or more panels.

Optionally, at least one of the shelf and the hook is configured to fold in a collapsed configuration of the barrier.

Optionally, the barrier includes a furniture connecting element fixed onto one of the one or more panels, wherein the furniture connecting element is configured to lock onto a portion of a bed, chair or table with a removable locking connection.

Optionally, the barrier includes a wall connecting element fixed onto one of the one or more panels, wherein the wall connecting element is configured to lock onto a mating connecting element on a wall, ceiling or floor of a room with a removable locking connection.

Optionally, the barrier includes a wall and wherein at least one of the one or more panels forms the wall of the barrier.

Optionally, the at least one of the one or more panels that form the wall of the barrier includes a seat defined along an upper edge, wherein the seat is configured to receive a ceiling panel.

Optionally, the wall is configured to fold along its height.

Optionally, the barrier includes a lever rotatably connected to a panel of the one or more panels, wherein the lever is configured to support the height of the wall in an expanded state while the lever is positioned in a first orientation and to allow collapsing of the wall while the lever is positioned in a second orientation.

Optionally, the barrier includes a ceiling and wherein at least one of the one or more panels forms the ceiling of the barrier.

Optionally, the barrier includes a floor and wherein at least one of the one or more panels is a floor of the barrier.

Optionally, the barrier includes a leg configured to stabilize one of the one or more panels that forms a wall of the barrier, wherein the leg is attached to the panel.

Optionally, the leg is rotatable and is configured to be positioned in a first orientation in the compact configuration of the barrier and in a second orientation in an expanded configuration of the barrier.

Optionally, the barrier includes a panel lock configured to lock the one or more panels in a collapsed state.

Optionally, the one or more panels are made from a rigid material.

Optionally, the one or more panels are made from transparent material surrounded by a frame.

Optionally, at least one panel of the one or more panels is formed from a different material, is a different size or a different shape that the other panels of the one or more panels.

Optionally, the barrier includes a roll of screen material that is fixed to a panel of the one or more panels wherein the screen material forms a screen over a portion of the barrier.

Optionally, the screen includes a screen window through which at least one of a physical examinations, diagnostic procedure and treatment may be performed, wherein the window on the one or panels and the screen window are aligned in an expanded state of the screen.

Optionally, the screen material is disposable and wherein the roll includes enough screen material for a plurality of screens.

Optionally, the barrier includes the one or more panels is a screen panel formed from flexible material that is rolled on a roller in a collapsed state and extend out from the roller in an expanded state.

Optionally, the roller is configured to be attached to a ceiling or a wall in a room.

Optionally, the roller is supported on legs and further comprising at least one rod configured to support the screen panel.

Optionally, the one or more panels are configured define an enclosure that at least partially encloses a bed, a chair, a table or a head of a care receiver.

Optionally, the barrier includes a plurality of windows.

Optionally, at least one of the plurality of windows has a different size or shape than another of the plurality of windows.

Optionally, one of the plurality of windows is configured to be fitted with an air filter, an air purifier, a ventilation system, microphone and a light fixture.

Optionally, the barrier includes a sleeve fitted onto the window.

Optionally, the sleeve includes an open end distal from the panel, wherein the opening is configured to be selectively closed.

Optionally, the sleeve includes an opening along a length of the sleeve, wherein the opening is configured to be selectively closed.

Optionally, the barrier includes a glove fitted onto the window.

Optionally, the barrier includes a window cover fitted on the window, wherein the window cover is configured to cover the window and is removably attached to the window.

Optionally, the window cover is sized to provide operative engagement between a care receiver on one side of the window cover and an imagining device on the opposite side of the window cover for imaging the care receiver.

Optionally, the window cover includes one or more of a sleeve and a glove.

Optionally, the window cover is configured to allow one or more of a sleeve and a glove to slide along the window.

Optionally, the window cover includes a connecting element along a length of one or more of the sleeve and the glove, wherein the connecting element is configured to replace one or more of the sleeve and the glove.

Optionally, the sleeve includes at least one of an opening along a length of the sleeve and an open end distal from the panel, both configured to be selectively closed.

According to an aspect of some example embodiments, there is provided a kit for constructing a barrier for use in one or more of physical examinations, diagnostics, treatments and recreation, the kit comprising: one or more panels, wherein at least one of the panels includes a window through which at least one of a physical examinations, diagnostic procedure and treatment may be performed, wherein the barrier includes at least one structural feature that is configured to reversibly collapse the barrier into a compact configuration while not in use or for transporting the barrier; one or more of a window cover, a sleeve and a glove configured to be mounted on the window.

Optionally, the one or more panels as described herein.

Optionally, the window cover as described herein.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE SEVERAL VIEW OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIGS. 1A and 1B are an examination booth and a window respectively as known in the prior art;

FIGS. 2A, 2B and 2C are schematic drawings of an example barrier shown from two opposite sides and in a partially folded state respectively, all in accordance with some example embodiments;

FIG. 3 is schematic perspective drawing of an example barrier supported against a wall, in accordance with some example embodiments;

FIG. 4 is a schematic drawing of an example leg integrated on a panel of an example barrier in accordance with some example embodiments;

FIG. 5 is a schematic drawing of an example barrier with example clasps that attach to a hospital bedframe, a dental chair or other platform associated with the care receiver in accordance with some example embodiments;

FIGS. 6A and 6B are schematic drawings of an example barrier including a ceiling, shown in an expanded state and in a partially folded state respectively, all in accordance with some example embodiments;

FIGS. 7A and 7B are schematic drawings of an example foldable ceiling shown in a flattened and partially folded state; both in accordance with some example embodiments;

FIG. 8 is a schematic drawing of an upper edge of an example barrier including a seat configured to receive a ceiling panel in accordance with some example embodiments;

FIGS. 9A, 9B, 9C, 9D, 9E and 9F are example window covers that are configured to be attached to a window on an example barrier in accordance with some example embodiments;

FIGS. 10A and 10B are schematic drawings of a doctor performing a physical examination through an example barrier and a patient receiving the physical examination through the example barrier respectively, both in accordance with some example embodiments;

FIG. 10C is a schematic drawing of a caregiver performing a physical examination through an example barrier in a seated position, in accordance with some example embodiments;

FIGS. 11A and 11B are schematic drawings of an example roll down barrier and an example roll up barrier respectively, both in accordance with some example embodiments;

FIGS. 12A and 12B are example box shaped barriers in accordance with some example embodiments;

FIGS. 13A and 13B is an example barrier configured to be folded into box shape, shown in an open and closed configuration both in accordance with some example embodiments; and

FIG. 14 showing an example barrier including a disposable screen in accordance with some example embodiments.

### DESCRIPTION OF SPECIFIC EMBODIMENTS OF THE INVENTION

The present invention, in some embodiments thereof, relates to a barrier for use in physical examinations, diagnostics and treatments and more particularly, but not exclusively, to a barrier that protects caregivers from contracting COVID-19 virus during physical examinations and treatments.

Before explaining at least one embodiment of the invention in detail, it is to be understood that the invention is not necessarily limited in its application to the details of construction and the arrangement of the components and/or methods set forth in the following description and/or illustrated in the drawings and/or the Examples. The invention is capable of other embodiments or of being practiced or carried out in various ways.

According to some example embodiments, the barrier includes one or more panels that are configured to collapse into compact dimensions for convenient storing while not in use and expanded as needed during use. Optionally, the barrier is constructed with modular panels that are configured to connect. The barrier may be collapsed for example based on folding or rolling. Optionally, folding may be with a hinge connection or based on a flexible connection between the panels, e.g. a flexible strap attached along one longitudinal edge to one panel and along an opposite longitudinal edge to another panel. In some example embodiments, in an expanded state, the connection between the panels is sealed to prevent respiratory droplets and/or air transfer from penetrating between the panels. The sealed connection may be provided with a flexible material, e.g. rubber or silicon. Optionally, a seal that is rigid is used. Optionally and preferably at least a portion of barrier is transparent so that a caregiver and care receiver, e.g. patient can see each other during the examination and/or treatment. Alternatively, the barrier is not transparent. In some example embodiments, the one or more panels are made from a rigid material, e.g. plexiglass (Poly(methyl methacrylate) (PMMA)), polycarbonate or glass. In other example embodiments, the one or more panels may be from a flexible material, e.g. nylon or polyvinyl chloride (PVC). Optionally, the barrier, e.g. one or more panels of the barrier may include a frame, e.g. an aluminum, plastic frame or rubber frame. PVC silicon. Optionally, the barrier does not include a frame.

According to some example embodiments, the barrier includes handles to facilitate lifting or transporting the barrier. Optionally, the handles attached to the panels and/or are integrated on a frame around one or more of the panels when a frame is included.

According to some example embodiments, one or more of the panels include a connector that is configured to connect to a matching connector and/or an existing structural element on a wall, bed, chair, awning, ceiling or floor for stabilizing the barrier. Optionally, the barrier is configured to be attached to a bedframe, e.g. a hospital bedframe, and/or to a dental chair. In some example embodiments, one or more of the panels include legs or other stabilizing structures. Optionally, the legs may be rotatable or otherwise collapsible for more compact storage. In some example embodiments, the barrier includes wheels or other sliding mechanism.

Optionally the barrier may be expanded to form a straight wall, may be semi-folded to form a ‘L’ shaped structure, a ‘U’ shaped structure, a ‘W’ shaped structure, or a polygonal shaped enclosure, e.g. a rectangular enclosure. Optionally, the barrier may form a self-supported structure in one or more semi-folded configurations. In some example embodiments, one or more of the panels are configured to form a ceiling or an awning of the barrier. Alternatively, the ceiling or awning is not be included.

According to some example embodiments, a barrier formed with a flexible panel may be collapsed by rolling. Optionally, the panel may be rolled on a spring actuated roller. The roller may be mounted on a ceiling, wall and/or may be integrated with a stand that is positioned on the floor. Optionally, the flexible panel is disposable, e.g. configured for one time use and the roller includes enough material to erect multiple barriers one after the other. Optionally, a blade or other cutting mechanism is integrated with the roller so that portion of the rolled that has been used can be removed and a new panel can be erected.

Example dimensions for a panel of a barrier is 1 m-2.5 m in height and 80 cm-1.5 meters, e.g. 1 m in width. Different heights and non-rectangular panels are also contemplated. For example, barrier configured to partially encompass a patient sitting on a chair may include a rectangular front panel and a pair of side panels that are sloped. Other sizes and shapes are contemplated.

At least one of the panels of the barrier includes a window, through which the caregiver can examine, diagnose and/or treat the care receiver. The windows may have different sizes and shapes depending on their dedicated function. Optionally, the window may have a standard size and shape and dedicated window covers positioned over the windows may provide different functionalities. Optionally, the window cover is configured to be selectively removed. Optionally, the window cover is disposable (optionally biodegradable). The window cover may be formed from flexible, rigid material, or a combination of both and may have a dedicated shape. The window cover may be formed from transparent material, opaque material or a combination of both. In some example embodiments, the window cover is removably fastened to the window. Optionally, a fabric hook and loop fastener, e.g. Velcro®, snaps, zipper or clasps may be used to fasten window cover to the window. Alternatively, the window cover is permanently fastened to the window or is integral to the panel.

Optionally, window cover includes one or more sleeves and/or may be fitted with one or more sleeves through which the caregiver can extend a hand or a device, e.g. stethoscope, thermometer, swab, syringe and ultrasound (US) transducer. Optionally, the window cover and/or sleeve may be configured to be movable along the window. Optionally, the one or more sleeves may be formed in the shape of a glove. Optionally, the covers include different size sleeves, e.g. diameter and length each suitable for different examination, treatment or physical setup. Optionally, one or more sleeves may include an opening through which a finger, hand or device may penetrate therethrough. Optionally, the opening may be selectively sealed and/or is configured to form a seal around the penetrating objects. More than one window with window cover may be included, e.g. a different heights and places.

The barrier may be modular structure including panels that may be clasped together. Modularity may provide different levels of protection and different functionality. Optionally, position of the window and/or size of the window may vary in different panels and an appropriate panel may be selected based on the examination, treatment and/or physical setup. In some example embodiments, a kit for the barrier may include panels with different types of windows. Optionally, the kit includes a ceiling structure. The ceiling structure may be formed from rigid material that is configured to be supported by walls barrier with an optionally sealed connection or a flexible material configured to be draped over walls of the barrier. Optionally, the kit includes a floor that may optionally provide a sealed connection with the walls of the barrier. In some example embodiments, the kit includes a plurality of window covers. The window covers may provide a variety of different functionalities.

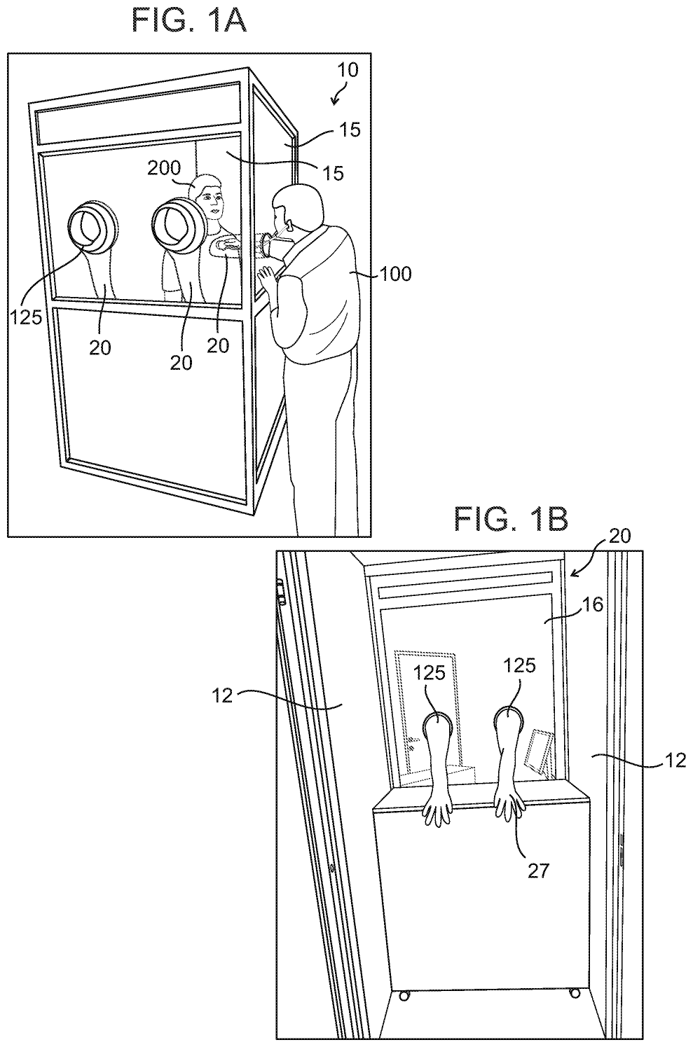

Reference is now made to FIGS. 1A and 1B showing an examination booth and a window respectively as known in the prior art. There have been a number of solutions proposed for examining a patient that is suspected of being infected with COVID-19. One solution that has been proposed includes admitting a patient 200 for examination by a doctor 100 while patient 200 is confined in a booth 10, e.g. similar in size and shape to a telephone booth and examining the patient 200 through flexible sleeves 20 integrated in opening 25 on a window panel 15 of booth 10. Both 10 has a plurality of disadvantages that may make it not suitable for widespread use. One disadvantage is associated with size and shape of booth 10. Booth 10 is inherently bulky and cannot be easily stored while not in use. Furthermore, due to its size and shape, booth 10 may be costly to deliver and assemble and may also be difficult to move to different areas of a medical facility based on changing needs. Booth 10 is not suitable to provide the care in the regular environment of the care receiver and/or care giver. Instead, the care receiver and caregiver is required to perform the care at booth 10. Another disadvantage of booth 10 is that it is not suitable for examining bed ridden patients or for performing examinations in a reclined state.

Another solution that has been proposed is to cover a service window with a window panel 16 that includes openings 25 fitted with gloves 27. In this solution panel 16 is fixedly attached to surrounding walls 12. One of the disadvantages with this setup is that it defines an examination area that is not transportable. A patient must arrive to the service window to get examined. In addition, the examination area is not suitable for examining bed ridden patients and/or for performing examinations while the patient is in a reclined state. Furthermore, window panel 16 requires surrounding walls on which it can be stabilized.

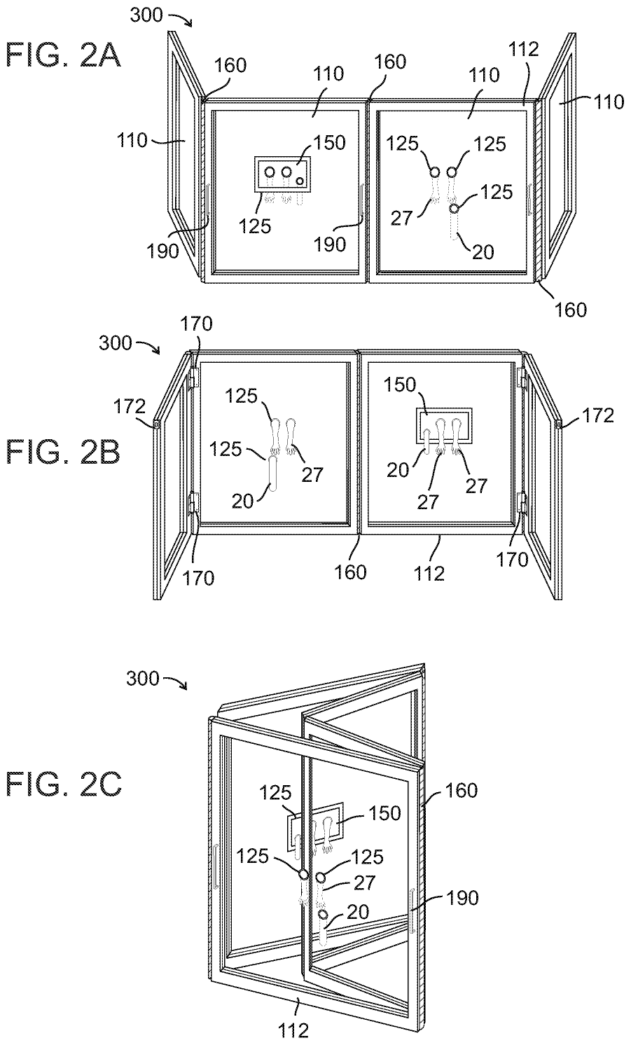

FIGS. 2A, 2B and 2C are schematic drawings of an example barrier shown from two opposite sides and in a partially folded state respectively, all in accordance with some example embodiments. According to some example embodiments, a barrier 300 includes a plurality of panels 110 that are attached with hinges 170. In other example embodiments, panels 110 may be connected by flexible or bendable material. In yet other example embodiments, barrier 300 is a single panel 110 that is folded along fold lines. Panels 110 include one or more windows 125 with window covers 150 through which a caregiver may examine and/or treat a care receiver. Panels 110 may be configured to form a partial enclosure and may not include a window 150 and may optionally have a different size than panel 110. Optionally one or more panels 110 provide for stabilizing barrier 300 so that barrier 300 is self-standing. Barrier may be used to enclose (or partially enclose) a caregiver or to enclose a person receiving the care. Window 125 and/or window cover 150 may be rectangular, circular or any other shape. Window cover 150 and/or may include one or more sleeves 20 and/or gloves 27. Optionally, a sleeve 20 or a glove 27 may be mounted directly on window 125.

In some example embodiments, a seal 160 is positioned between the connecting panels 110 of barrier 300 and is configured to seal a gap formed between panels 110. Seal 160 may be rubber, silicon or other flexible material that may provide protection against respiratory droplets that may otherwise penetrate between panels. In some example embodiments, seal 160 is a strap that connects panels 110 and hinge 170 is not needed. Optionally, seal 160 may be formed from a rigid material. Seal 160 may for example be removably attached to the panels 110. Optionally, one or more of panels 110 include a wall connecting element 172 configured to connect to a mating element e.g., a screw or bolt that is on the wall. According to some example embodiments, barrier 300 may be folded into a flat configuration for storage. Optionally, barrier 300 may be folded in a box configuration, triangular configuration or other configuration. Optionally, barrier 300 includes one or more handles 190 that facilitate folding and moving barrier 300. According to some example embodiments, barrier 300 may be expanded into different shaped structures, e.g., that may define partial or full enclosures. Optionally, barrier 300 may be expanded to form a ‘U’ shaped structure, an ‘L’ shaped structure, a ‘W’ shaped structure, a polygonal structure or a straight wall.

According to some example embodiments, a portion of barrier 300 is transparent so that a caregiver and patient may see each other. Optionally, panels 110 are opaque. Optionally, panels 110 include a frame 112, e.g. aluminum profiles or plastic profiles.

According to some example embodiments, panels 110 include one or more windows 125. In some example embodiments, window 125 includes a window cover 150 including one or more sleeves 20 and/or gloves 27 or window 125 may directly covered with sleeve 20 and/or glove 27. Optionally, sleeves 20 and/or gloves 27 may be configured to be removable from window cover 150 and replaced. Window cover may be rectangular, circular or any other shape. Window cover 150 may be flexible and sleeve 20 and/or glove 27 may be extendable in both directions with respect to barrier 300. Windows 125 may be positioned at different heights and may be covered with different types of covers 150, sleeves 20 and/or gloves 27. Optionally, each window 125 provides a dedicated functionality. Window cover 150 and/or sleeves 20 may be permanent or replaceable. In some example embodiments, cover 150, sleeves 20 and/or gloves 27 may be disposable. Optionally, sleeve 20 or glove 27 may be directly mounted on window 125. Optionally, window cover 150 includes a plurality of leaves through which a person can extend an instrument, a hand, arm, foot or leg.

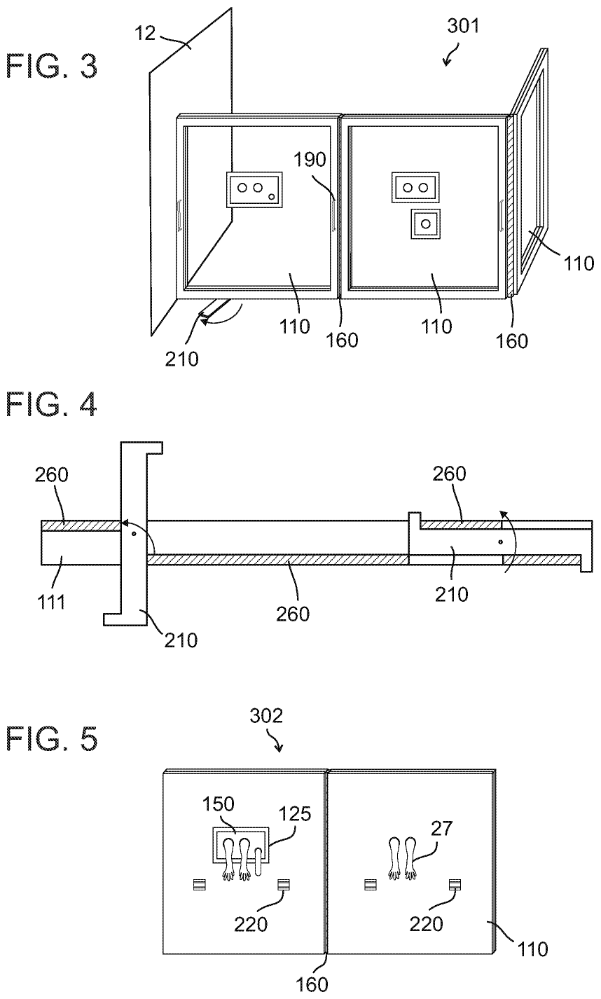

Reference is now made to FIG. 3 showing a schematic perspective drawing of an example barrier supported against a wall, in accordance with some example embodiments. According to some example embodiments, a barrier 301 may be configured to form an ‘L’ shaped structure that may optionally be positioned against a wall 12. In some example embodiments, barrier 301 includes one or more legs 210. Optionally, barrier 301 is self-standing with leg 210. In some example embodiments, barrier 301 may include a connecting element that attaches to a counterpart connecting element on wall 12. Optionally, barrier 301 formed in an ‘L’ shaped structure may be free standing and may not be positioned against wall 12. Optionally, barrier 301 formed in an ‘L’ shaped structure may positioned to partially encompass a bed or an examination platform on which a patient may recline, sit and/or stand.

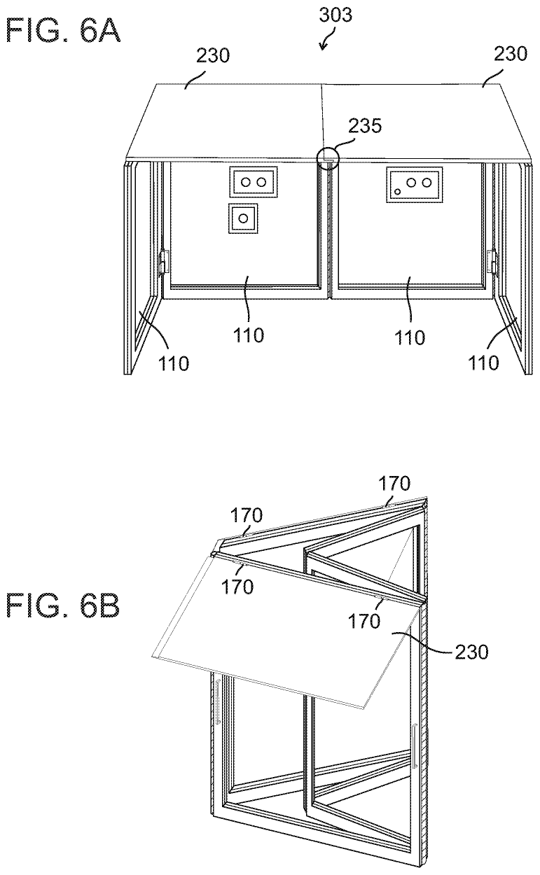

Reference is now made to FIG. 4 showing a schematic drawing of an example leg integrated on a panel of an example barrier in accordance with some example embodiments. According to some example embodiments, a bottom edge 111 of one or more panels 110 includes one or more rotatable legs 210. Optionally, leg 210 may be aligned with edge 111 (manually aligned) while the barrier is in a collapsed state and may be rotated, e.g., rotated 90 degrees to provide leg support for the barrier when expanded. In some example embodiments, bottom edge 111 includes a seal 160 that is configured to maintain a seal between barrier 301 and supporting floor while leg 210 is rotated to extend out from edge 111.

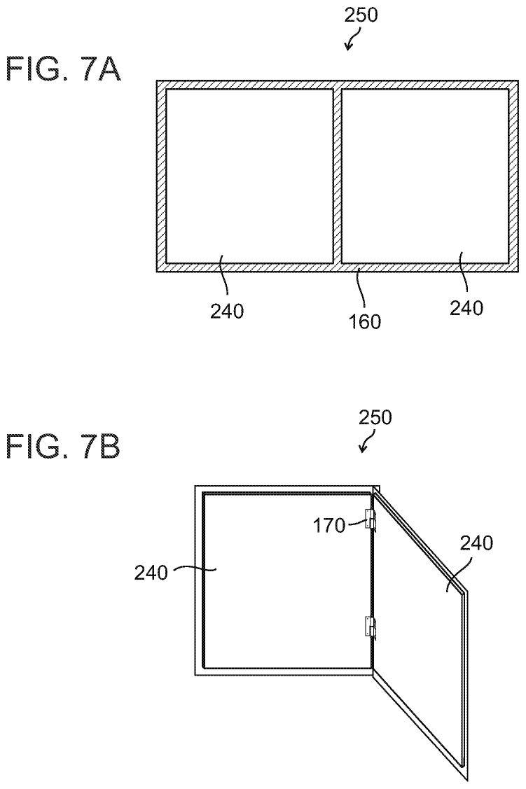

FIG. 5 is a schematic drawing of an example barrier with example clasps that attach to a hospital bedframe, a dental chair or other platform associated with the care receiver in accordance with some example embodiments. According to some example embodiments, a barrier 302 is configured to be attached to a bed, chair or other platform for examining, diagnosing and/or treating a care receiver. According to some example embodiments, barrier 302 includes a connecting element 220 fixed on panel 110. Optionally, connecting element 220 is a clasp configured clasp on (or lock onto) a portion of an examination bed, other bed, a chair, a table or other furniture. Optionally, a bar on a bed or a chair is snapped into connecting element 220 while in use and may be pulled when not needed. Optionally, barrier 302 is configured to be raised above the floor while attached to the bed and is fully structurally supported with clasps 220.

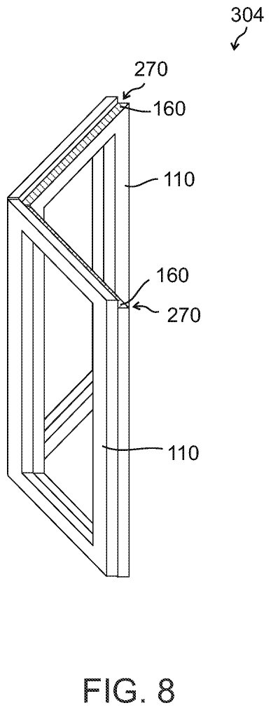

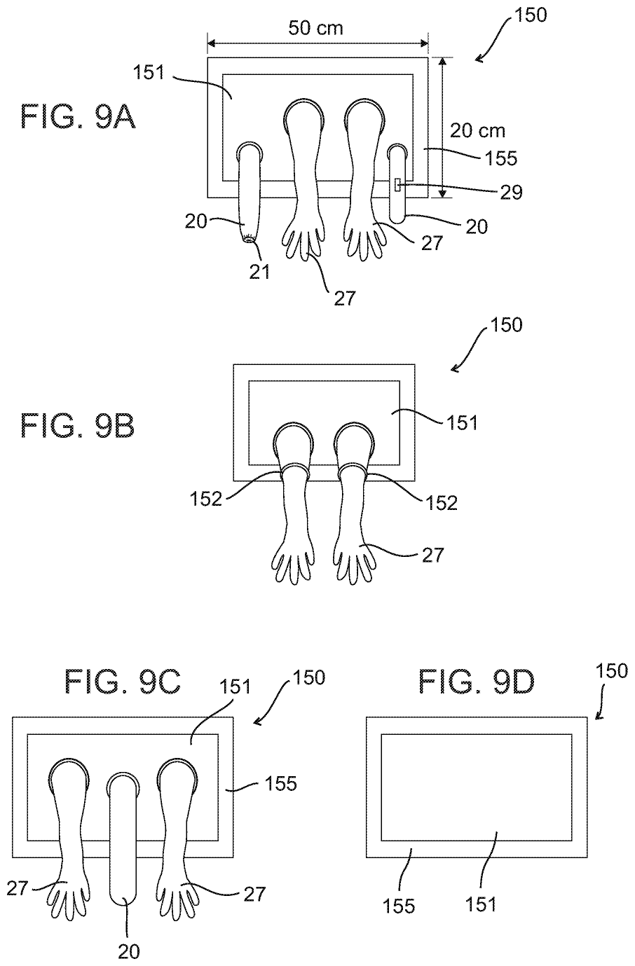

Reference is now made to FIGS. 6A and 6B showing schematic drawings of an example barrier including a ceiling, shown in an expanded state and in a partially folded state respectively, all in accordance with some example embodiments. According to some example embodiments, a barrier 303 includes one or more wall panels 110 and additionally includes one or more ceiling panels 230. In some example embodiments, ceiling panels 230 are connected to wall panels 110 by a hinged connection 170 or other flexible connection (FIG. 7B) based on which panels 110 are pivotally connected. Ceiling panels 230 may fully or partially cover an enclosure defined by barrier 303. Optionally each of a pair of ceiling panels 230 include lips 235 that configured to fit one to another to provide a sealed seam between ceiling panels 230. Ceiling panels 230 may optionally be formed from light weight material that may be transparent or opaque. According to some example embodiments, ceiling panels 230 may be folded in a flat configuration while stored in a collapsed state. Optionally, ceiling panels 230 may be disengaged from wall panels 110. Optionally, ceiling panel 230 may be a single panel attached to one of wall panels 110 or may be a single panel that is not attached to any of wall panels 110 but rather placed over wall panels 110. Optionally, ceiling panel 230 is configured to be folded. In some example embodiments, barrier 303 may similarly include one or more floor panels that is either attached to wall panels 110 or separate from wall panels 110.

FIGS. 7A and 7B are schematic drawings of an example foldable ceiling shown in a flattened and partially folded state; both in accordance with some example embodiments. According to some example embodiments, a ceiling 250 may be formed from one or more panels 240 that is configured to be positioned over a barrier, e.g. barrier 300 (FIGS. 2A, 2B and 2C) or barrier 301 (FIG. 3). In some example embodiments, ceiling 250 includes at least two ceiling panels 240 that are configured to fold. Optionally, ceiling panels 240 are connected with one or more hinges 170. In alternate examples ceiling 250 is a single panel that is foldable along one or more fold lines. Optionally, edges of ceiling panels 240 including a sealing material 160, e.g. rubber or silicon that is configured to provide sealed connection with walls of a barrier when placed thereon. Optionally, a floor for a barrier, e.g. barrier 300 (FIGS. 2A, 2B and 2C) or barrier 301 (FIG. 3) may have a same or similar construction as ceiling 250.

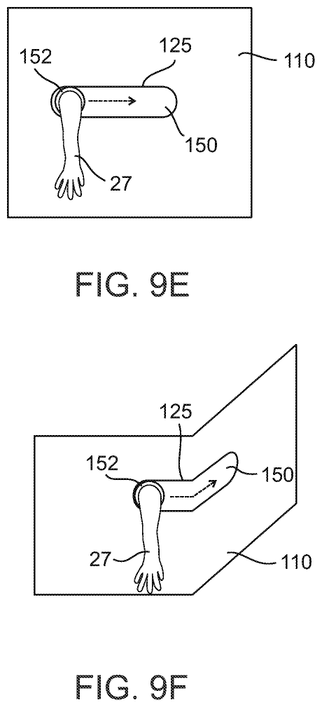

FIG. 8 is a schematic drawing of an upper edge of an example barrier including a seat configured to receive a ceiling panel in accordance with some example embodiments. According to some example embodiments, a barrier 304 includes a seat 270 formed on an upper edge of front panels 110 that is configured to receive a ceiling panel, e.g. ceiling panel 250 (FIGS. 7A and 7B). Optionally, seat 270 includes a sealing material, e.g. rubber or silicon to provide a sealed connection withs the ceiling panel when positioned thereon.

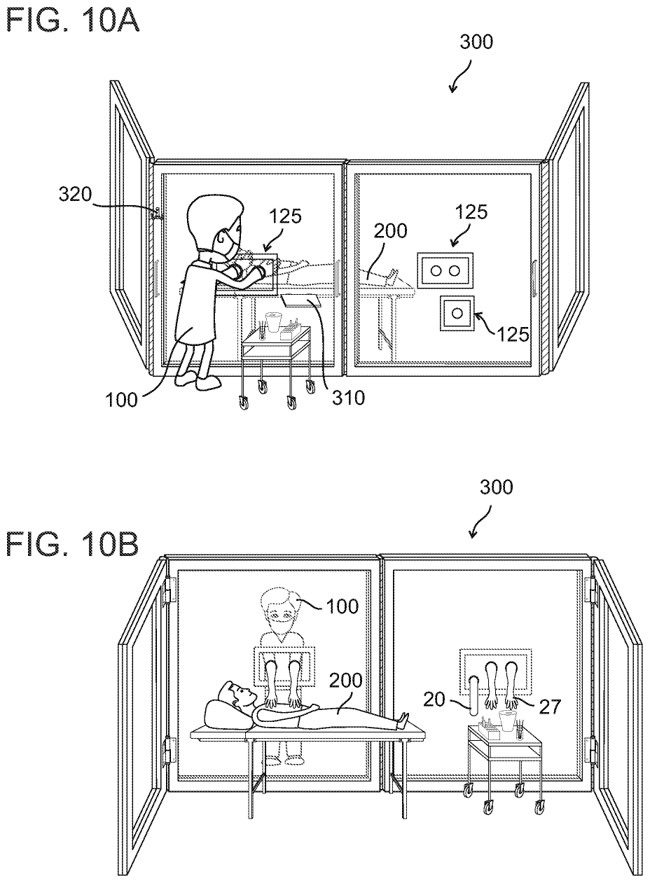

Reference is now made to FIGS. 9A, 9B, 9C, 9D, 9E and 9F showing 9F are example window covers that are configured to be attached to a window on an example barrier in accordance with some example embodiments. According to some example embodiments, a barrier as described herein includes one or more windows that may be covered with different window covers 150 based on the type of examination and/or treatment being performed. According to some example embodiments, window cover 150 includes a base 151 with a frame 155 that can be fixed around a window. Optionally frame 155 includes a fastener, e.g. fabric hook and loop fastener (Velcro®) that fastens onto a counterpart fastener around the window. Alternatively, frame 155 may fit into the window, e.g. with or without a dedicated connecting mechanism. Base 151 may be formed from rigid or flexible material, e.g. bendable or elastic material that is either transparent, opaque or semi-transparent. Optionally, window cover frame 155 may be permanently fixed to the window and one or more portions of window cover 150 may be replaceable. It is noted that although window cover 150 is depicted herein as a rectangular cover, other shapes for window cover 150 as well as for windows on the barrier are also contemplated.

In some example embodiments, one or more sleeves 20 or gloves 27 may extend from base 151. In some example embodiments, one or more sleeves, e.g. tubular sleeves 20 or gloves 27 may be integral with base 151 and formed from a same material. Optionally, cover 150 may be configured for one time use, e.g. may be disposable. Optionally, material of cover 150 is biodegradable. Optionally, one or more of tubular sleeves 20 may include an opening 21 through which a swab or an instrument may protrude. Optionally, the opening includes an elastic band that at least partially seals the opening. Optionally, opening 21 is sized to allow a person to expose a hand, arm, and/or leg therethrough. Optionally, one or more of tubular sleeves 20 may include an opening 29 through which a portion of a body part of the care receiver may be exposed. Optionally, opening 29 may be suitable for exposing an arm for blood transfusion, performing a blood test, or examining a wound.

Referring now to FIG. 9B, in some example embodiments, one or more of tubular sleeves 20 and/or gloves 27 are connected to base 151 with a dedicated connector 152. Optionally, connector 152 may be selectively disengaged to replace one of the sleeves, e.g. tubular sleeve 20 and/or glove 27. Optionally, one or more of tubular sleeves 20 and gloves 27 may be configured for one time use. Optionally, one or more of tubular sleeves and gloves configured for one time use may be positioned over tubular sleeves 20 and/or gloves 27 and optionally connected with connector 152.

Referring now to FIG. 9D, in some example embodiments, window cover 150 does not include any sleeves, e.g. tubular sleeves 20 or gloves 27 and is configured to seal one or more windows on the barrier that may not be in use. Optionally, window cover 150 may be configured to provide performing an X-ray or other diagnostic procedure therethrough. Optionally, one or more of window cover 150 includes an air filter or air purifier.

Referring now specifically to FIGS. 9E and 9F, in some example embodiments, window cover 150 is configured to providing shifting glove 27 (or other sleeve) to a desired position along wall panel 110. Optionally, window cover 150 may also provide shifting around a bent corner. Optionally, window cover 150 maintains a seal over window 125 as glove 27 slides along window 125. Optionally, window cover 150 is formed with flexible material that bunches up and extends due to sliding of glove 27. Optionally, window cover 150 slides along a railing on panel 110.

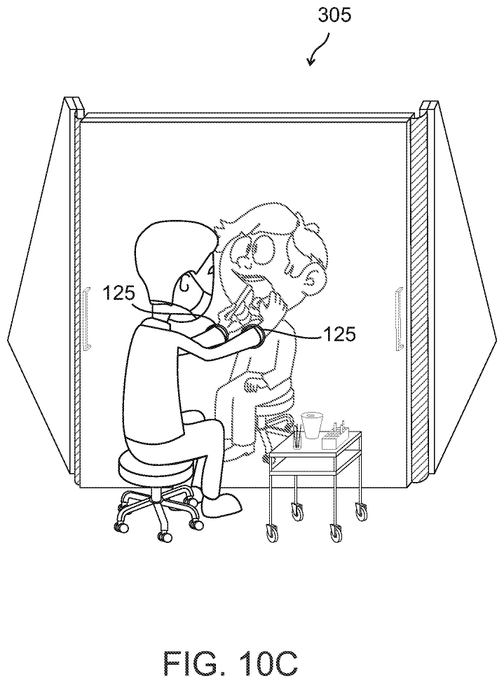

Reference is now made to FIGS. 10A and 10B showing schematic drawings of a doctor performing a physical examination through an example barrier and a patient receiving the physical examination through the example barrier respectively, both in accordance with some example embodiments. In some example embodiments, a caregiver 100 may examine or treat a patient 200 that is enclosed within barrier 300 while the caregiver 100 is outside the enclosure defined by barrier 300. Alternatively, caregiver 100 may stand within the enclosure and the patient 200 may be outside the enclosure. Optionally, examination, diagnosing and/or treatment may be performed while patient 200 is sitting, standing or lying down. Optionally, barrier 300 includes a plurality of windows 125 that accommodate reaching the patient in a plurality of different postures and/or reaching different areas on patient 200. According to some example embodiments, caregiver 100 inserts devices, e.g. instruments for examination and/or treatment through tubular sleeves 20 or gloves 27. Optionally, patient 200 inserts an arm, hand, foot or leg through one or more tubular sleeves 20, e.g. same or different tubular sleeves used by caregiver 100. Optionally, one or more devices or items are positioned on the side of the patient 200 and the caregiver 100 can grasp the devices or items with gloves 27. In some example embodiments, barrier 300 includes one or more shelves 310 and/or hooks 320 on which instruments, garments may be positioned. Optionally, shelf 310 is foldable.

Reference is now made to FIG. 10C showing a schematic drawing of a caregiver performing a physical examination through an example barrier in a seated position, in accordance with some example embodiments. According to some example embodiments, a barrier 305 is configured to at least partially encompass a person sitting on a chair. Optionally, dimensions of barrier 305 may be smaller as compared to a barrier configured for a bed or an examination in a standing posture. According to some example embodiments, positioning of the windows 125 is defined to accommodate the examination or therapy that is being performed.

In some example embodiments, barrier 305 may be used for recreation purposes. For example barrier 305 may be used in schools to allow students to play a board game together. Optionally, barrier 305 includes openings through which a person can extend their feet.

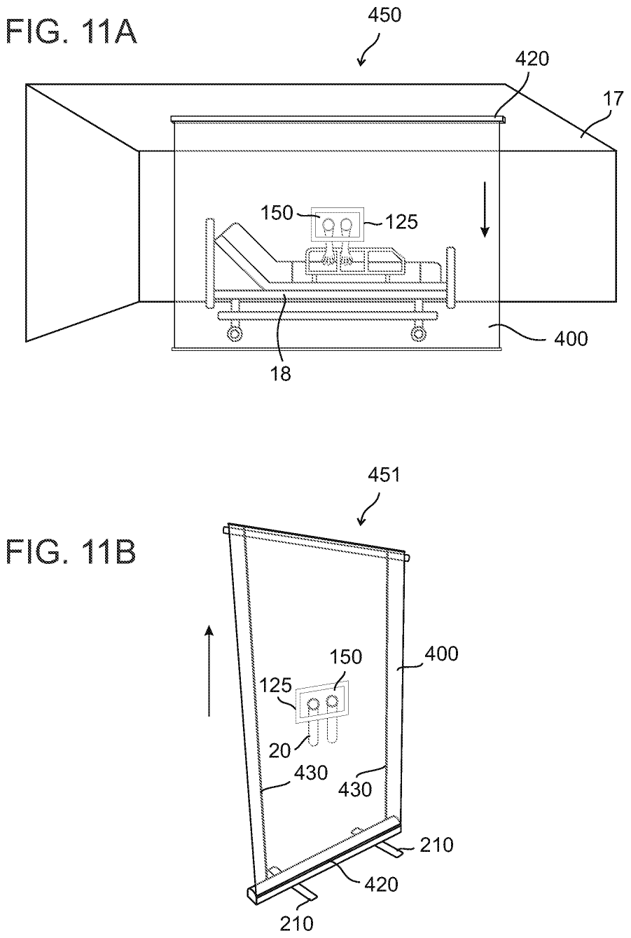

Reference is now made to FIGS. 11A and 11B showing schematic drawings of an example roll down barrier and an example roll up barrier respectively, both in accordance with some example embodiments. According to some example embodiments, a barrier 450 (FIG. 11A) is in the form of a flexible pull down screen 400 that may be rolled and/or unrolled with a roller 420. Optionally, roller 420 may be connected to a ceiling 17 and pulled down when needed. Optionally, roller 420 may be connected to a wall and pulled out and attached to an opposite facing wall when needed. According to some example embodiments, screen 400 includes one or more windows 125 one which an examination cover 150 may be positioned described herein. Optionally, a sleeve or glove may be positioned over a window 125. In some example embodiments, barrier 450 is sized to extend over a length of a hospital bed 18 or may extend over a portion thereof. Optionally, roller 420 includes enough screen material for more than one screen 400 and a caregiver can tear off and discard the portion of screen 400 that was used and then pull out another screen 400 from roller 420, e.g. screen 400 may be disposable. Optionally, each screen 400 includes at least one window 125. Alternatively, window 125 may be opened or formed by the caregiver at the time of the examination.

According to some example embodiments, a barrier 451 (FIG. 11B) is in the form of a flexible pull-up screen that may be rolled and/or unrolled with a roller 420 positioned near the floor, e.g. similar to a pull-up poster. Optionally, one or more rods 430 may be configured to support screen 400 in an upright position when pulled out. Optionally, a barrier 451 is self-supported with legs 210. According to some example embodiments, screen 400 includes one or more windows 125 one which an examination cover 150, sleeve or glove may be positioned as described herein. Optionally, roller 420 includes enough screen material for more than one screen 400 and a caregiver can tear off and discard the portion of screen 400 that was used and then pull out another screen 400 from roller 420. Optionally, each screen 400 includes at least one window 125. Alternatively, window 125 may be opened or formed by the caregiver at the time of the examination. In alternate examples, screen 400 may be removably connected between two poles and the barrier is the poles together with screen 400.

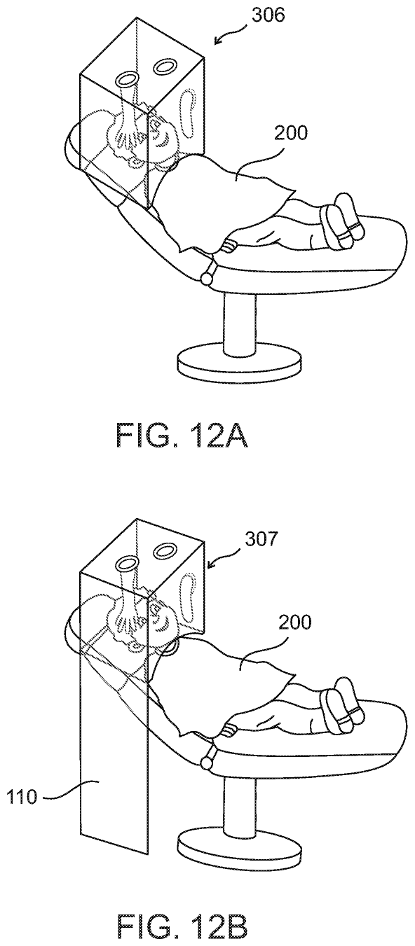

Reference is now made to FIGS. 12A and 12B show example are example box shaped barriers in accordance with some example embodiments. According to some example embodiments, barrier 306 and/or barrier 307 may be configured to form an enclosure over a portion of a patient's (or care receiver's) body. Optionally, barrier 306 is configured to fit over a head of patient 200 and may be configured for dental examination, diagnosing and/or treatment. In some example embodiments, barrier 306 is shaped to be supported on a dental chair. In some example embodiments, barrier 307 may include one or more panels 110 that extend to the floor. In some example embodiments, each of barriers 306 and 307 are foldable.

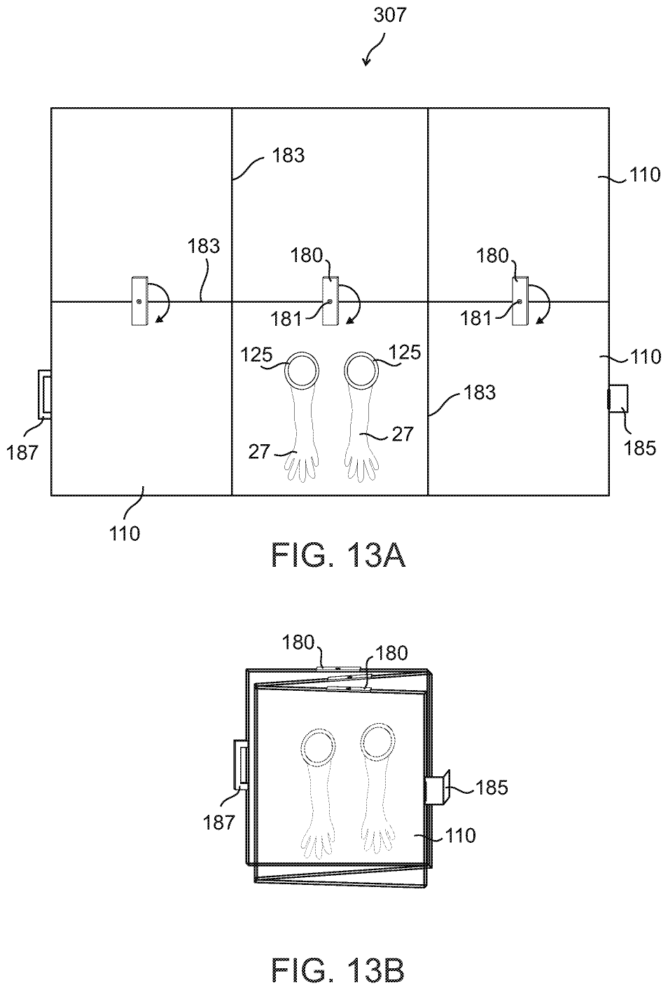

Reference is now made to FIGS. 13A and 13B showing an example barrier configured to be folded into box shape, the barrier shown in an open and closed configuration both in accordance with some example embodiments. According to some example embodiments, a barrier 307 may be collapsed to a smaller configuration based on folding barrier 307 along its height and its width. Barrier 307 may include a plurality of panels 110 that are connected, e.g. by a hinge or other flexible connection or may be a single panel 110 that is configured to fold along fold lines 183 formed barrier 307. According to some example embodiments, one or more levels 180 may be positioned on panels 110 to support erection of barrier 307 from a folded to a straight position. Optionally, levers 180 are pivotally attached to panels 110 of barrier 307, e.g. levels 180 rotate about pin 181.

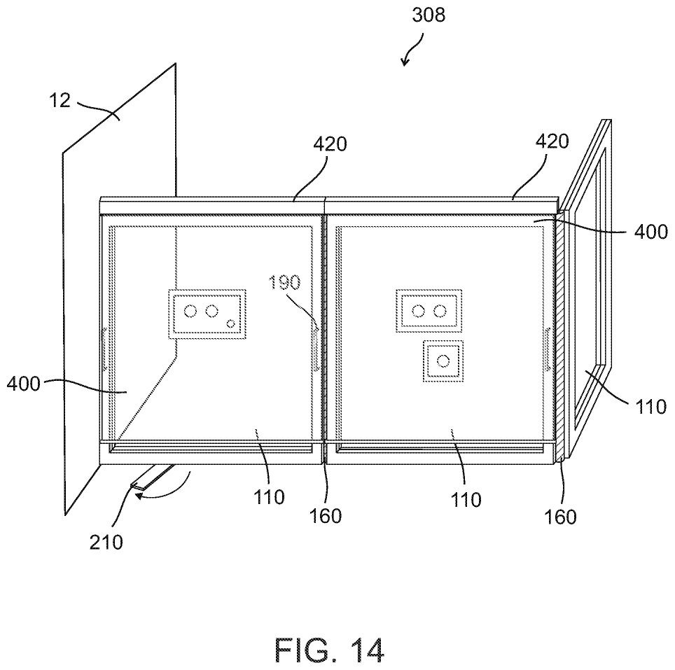

Reference is now made to FIG. 14 showing an example barrier including a disposable screen in accordance with some example embodiments. According to some example embodiments, a barrier 308 includes one or more panels 110 and additionally include a roller 420 that houses a roll of screen material 400. Optionally screen material 400 may be pulled down to provide additional protection. Optionally, screen material 400 may be disposable. Optionally, roller 420 is mounted on one or more of panels 110. Roller 420 may be mounted on either or both sides of panels 110. Optionally, barrier 308 may be folded in the form of a box or suitcase and may be suitable for transporting to different locations, e.g. by car or by ambulance. Optionally, barrier 308 includes a lock 185 for locking barrier 308 in a collapsed state. Optionally, barrier 308 includes a handle 187 for carrying barrier 308 in a collapsed state.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination or as suitable in any other described embodiment of the invention. Certain features described in the context of various embodiments are not to be considered essential features of those embodiments, unless the embodiment is inoperative without those elements.

In addition, any priority document(s) of this application is/are hereby incorporated herein by reference in its/their entirety.

1-46. (canceled)

47. A barrier for physical examinations, diagnostics, treatments and/or recreation, the barrier comprising one or more panels, wherein at least one of the panels includes at least three windows through which the at least one of a physical examinations, diagnostic procedure and treatment may be performed, wherein the at least three windows includes two windows on which gloves are mounted for receiving hands and one window on which a sleeve is mounted for receiving an instrument to be operated with the hands received in the other two windows, the barrier further comprising a window cover fitted on one of the at least three windows, wherein the window cover is configured to cover the one of the at least three windows and is removably attached to the one of the at least three windows, wherein a further sleeve or further glove is mounted on the window cover and wherein the window cover is configured to allow the further mounted sleeve or glove to slide along the one of the at least three windows.

48. The barrier according to claim 47, wherein the one window on which the sleeve is mounted is positioned between the two windows on which the gloves are mounted.

49. The barrier according to claim 47, wherein the sleeve includes an open end distal from the panel, wherein the open end is configured to be selectively closed, or includes an opening along a length of the sleeve, wherein the opening is configured to be selectively closed.

50. The barrier according to claim 47, wherein one of the at least three windows configured for receiving the instrument has a different size and/or shape than another of the at least three windows configured to receive the hands.

51. The barrier according to claim 47, wherein the at least three windows additionally includes a window configured to be fitted with an air filter, an air purifier, a ventilation system, microphone or a light fixture.

52. The barrier according to claim 47, wherein the barrier includes at least one structural feature that is configured to reversibly collapse the barrier into a compact configuration while not in use or for transporting the barrier.

53. The barrier according to claim 52, wherein the at least one structural feature is selected from a group consisting of:
a fold line in the panel and wherein the panel of the one or more panels is configured to be folded along fold lines in the panel
a hinge connecting a pair of panels of the one or more panels and wherein the barrier is configured to fold along the hinge connection
a flexible strap connecting a pair of panels of the one or more panels and wherein the barrier is configured to fold along the flexible strap; and
a locking mechanism and wherein the one or more panels are modular elements are configured lock onto one another with a removable locking connection.

54. The barrier according to claim 47, comprising a seal configured to seal a gap in a connection between the one or more panels.

55. The barrier according to claim 47, comprising a handle fixedly attached to at least one of the one or more panels based on which the barrier may be manually handled.

56. The barrier according to claim 47, comprising at least one of a hook and a shelf fixedly attached to at least one of the one or more panels, wherein at least one of the shelf and the hook is configured to fold in a collapsed configuration of the barrier.

57. The barrier according to claim 47, comprising a furniture connecting element fixed onto one of the one or more panels, wherein the furniture connecting element is configured to lock onto a portion of a bed, chair or table with a removable locking connection.

58. The barrier according to claim 47, comprising a wall connecting element fixed onto one of the one or more panels, wherein the wall connecting element is configured to lock onto a mating connecting element on a wall, ceiling or floor of a room with a removable locking connection.

59. The barrier according to claim 47, wherein the barrier includes a wall and wherein at least one of the one or more panels forms the wall of the barrier, wherein the at least one of the one or more panels that form the wall of the barrier includes a seat defined along an upper edge, wherein the seat is configured to receive a ceiling panel and wherein the wall is configured to fold along its height.

60. The barrier according to claim 59, comprising a lever rotatably connected to a panel of the one or more panels, wherein the lever is configured to support the height of the wall in an expanded state while the lever is positioned in a first orientation and to allow collapsing of the wall while the lever is positioned in a second orientation.

61. The barrier according to claim 47, wherein the barrier includes a ceiling and wherein at least one of the one or more panels forms the ceiling of the barrier.

62. The barrier according to claim 47, wherein the barrier includes a floor and wherein at least one of the one or more panels is a floor of the barrier.

63. The barrier according to claim 47, comprising a leg configured to stabilize one of the one or more panels that forms a wall of the barrier, wherein the leg is attached to the panel, wherein the leg is rotatable and is configured to be positioned in a first orientation in the compact configuration of the barrier and in a second orientation in an expanded configuration of the barrier.

64. The barrier according to claim 47, comprising a panel lock configured to lock the one or more panels in a collapsed state.

65. The barrier according to claim 47, wherein the one or more panels are made from a rigid material.

66. The barrier according to claim 47, wherein the one or more panels are made from transparent material surrounded by a frame.

67. The barrier according to claim 47, wherein at least one panel of the one or more panels is formed from a different material, is a different size or a different shape that the other panels of the one or more panels.

68. The barrier according to claim 47, comprising a roll of screen material that is fixed to a panel of the one or more panels wherein the screen material forms a screen over a portion of the barrier, wherein the screen material is disposable and wherein the roll includes enough screen material for a plurality of screens.

69. The barrier according to claim 47, wherein the one or more panels are configured define an enclosure that at least partially encloses a bed, a chair, a table or a head of a care receiver.

70. A barrier for one or more of physical examinations, diagnostics, treatments and recreation, the barrier comprising one or more panels, wherein at least one of the panels includes at least three windows through which the at least one of a physical examinations, diagnostic procedure and treatment may be performed, wherein the at least three windows includes two windows on which gloves are mounted for receiving hands and one window on which a sleeve is mounted for receiving an instrument to be operated with the hands received in the other two windows, the barrier, comprising a roll of screen material that is fixed to a panel of the one or more panels wherein the screen material forms a screen over a portion of the barrier, wherein the screen material is disposable and wherein the roll includes enough screen material for a plurality of screens, the screen including a screen window through which at least one of a physical examinations, diagnostic procedure and treatment may be performed, wherein the window on the one or panels and the screen window are aligned in an expanded state of the screen.

71. A barrier for one or more of physical examinations, diagnostics, treatments and recreation, the barrier comprising one or more panels, wherein at least one of the panels includes at least three windows through which the at least one of a physical examinations, diagnostic procedure and treatment may be performed, wherein the at least three windows includes two windows on which gloves are mounted for receiving hands and one window on which a sleeve is mounted for receiving an instrument to be operated with the hands received in the other two windows, and wherein the one or more panels include a screen panel formed from flexible material that is rolled on a roller in a collapsed state and extends out from the roller in an expanded state, wherein the roller is configured to be attached to a ceiling or a wall in a room, and wherein the roller is supported on legs and further comprises at least one rod configured to support the screen panel.
